(19) United States
(12) Patent Application Publication
(10) Pub. No.: US 2021/0100821 A1
(43) Pub. Date: Apr. 8, 2021

(54) STABLE LYOPHILIZED COMPOSITION OF CYCLOPHOSPHAMIDE

(71) Applicant: INTAS PHARMACEUTICALS LTD., Ahmedabad, Gujarat (IN)

(72) Inventors: Aditya PATEL, Ahmedabad (IN); Kerul PATEL, Ahmedabad (IN); Dhaval DADHANIYA, Ahmedabad (IN)

(21) Appl. No.: 16/971,179

(22) PCT Filed: Feb. 26, 2019
(86) PCT No.: PCT/IB2019/051515

§ 371 Date: Aug. 19, 2020

(30) Foreign Application Priority Data
Feb. 26, 2018 (IN) IN201821007142

(51) Int. Cl. A61K 31/675; A61K 9/19; A61K 47/26

(52) U.S. Cl. CPC A61K 31/675; A61K 47/26; A61K 9/19

(57) ABSTRACT

This present invention relates to a stable lyophilized composition of Cyclophosphamide. The said stable lyophilized composition of Cyclophosphamide provides an improved moisture content than the lyophilized Cyclophosphamide compositions obtained by the conventional lyophilization method. Further, the invention relates to a process for preparation of the said stable lyophilized composition of Cyclophosphamide, wherein the said process comprises controlled lyophilization with freezing and annealing at a temperature above 0° C.

### RELATED APPLICATIONS

This application is related to Indian Provisional Application 201821007142 filed 26 Feb. 2018 and is incorporated herein in its entirety.

### FIELD OF THE INVENTION

This invention relates to a stable lyophilized composition of Cyclophosphamide. The said stable lyophilized composition of Cyclophosphamide provides an improved moisture content than the lyophilized Cyclophosphamide compositions obtained by the conventional lyophilization method. Further, the invention relates to a process for preparation of the said stable lyophilized composition of Cyclophosphamide.

### BACKGROUND OF THE INVENTION

Cyclophosphamide, chemically known as (RS)—N, N-bis (2-chloroethyl)-1,3,2-oxazaphosphinan-2-amine 2-oxide, is a synthetic antineoplastic drug, and it is used in the treatment of malignant diseases and nephrotic syndrome.

Like other nitrogen mustards, cyclophosphamide degrades in the aqueous solutions, and hence it is available as a lyophilized powder, namely CYTOXAN®. The previously available lyophilized powder comprised of Cyclophosphamide monohydrate and mannitol, however the product is discontinued now. Further, Cyclophosphamide monohydrate is commercially available neat i.e. without any bulking agent. However, this product is difficult to dissolve and hence difficult to reconstitute.

The U.S. Pat. No. 5,418,223 discloses a process for preparation of lyophilized composition of cyclophosphamide. The process comprises sequential steps: (i) freezing a bulk solution containing cyclophosphamide and bulking agent, (ii) removing first portion of the water from frozen solution, (iii) melting of cake to produce supersaturated solution of cyclophosphamide and bulking agent, (iv) precipitating supersaturated solution of cyclophosphamide as a hydrated polymorph, (v) re-freezing the solution containing precipitated cyclophosphamide, and (vi) removal of water not bound to cyclophosphamide. Further, the removal of water is conducted at a temperature less than −15° C. and a pressure less than 8000 microns.

The PCT Application No. WO2014068585 discloses a composition comprising lyophilized cyclophosphamide monohydrate and its process for preparation wherein the lyophilization process is carried out without a rehydration step. Further, lyophilization is carried out in the presence of solvent or mixtures of solvents. The freezing is performed at temperatures below −12° C., and the drying is performed at temperatures between −50° C. and −5° C. The vacuum used for drying steps is between 10 mtorr and 1500 mbar. Furthermore, the process involves at least one annealing step with temperature ranging between −10° C. and −90° C. during freezing and drying steps. The water content in the finished product is between 5.5% W/W to 8.5% W/W.

Although lyophilized Cyclophosphamide product is well studied in the literature, there are formulation issues related to stability, reconstitution and water content. The previously available lyophilized product was difficult to reconstitute and it sometimes converts to yellow cake due to stability concern. Therefore, the inventors of the present invention have developed a stable lyophilized Cyclophosphamide composition that is easier to reconstitute and provides improved moisture content.

Further, there exists a need for a controlled lyophilization process for preparation of such lyophilized compositions.

### OBJECTS OF THE INVENTION

The main objective of the present invention is to provide a stable lyophilized cyclophosphamide composition that is easier to reconstitute and have improved moisture content than the currently available lyophilized composition of Cyclophosphamide.

Another object of the present invention is to provide a stable lyophilized composition of Cyclophosphamide prepared by a process comprising controlled lyophilization, wherein the annealing step is done at a temperature above 0° C.

### SUMMARY OF THE INVENTION

In a first embodiment, the present invention relates to a stable lyophilized cyclophosphamide composition that can be reconstituted in less than about 120 seconds and having moisture content of not less than about 3.5% W/W.

In another embodiment, the present invention relates to a stable lyophilized composition of Cyclophosphamide prepared by a process comprising controlled lyophilization, wherein the annealing step is done at a temperature from about 2° C. to 15° C.

Another embodiment, the present invention relates to a process for preparation of a stable lyophilized cyclophosphamide composition; wherein the process comprises the steps of:
i) Preparing a bulk solution comprising cyclophosphamide, mannitol, and water for injection,
ii) Filling the bulk solution into glass vials, which are then rubber stoppered,
iii) Loading the vials of step ii) into Lyophilizer at a temperature of about 5 to 25° C.,
iv) Performing the freezing step at a controlled temperature in the Lyophilizer, wherein annealing step is performed at a temperature from about 2° C. to 15° C., followed by freezing at about −45° C.
v) Setting the condenser temperature to −60° C. along with vacuum at 200 mtorr,
vi) Performing the primary drying at −18° C. with ramp for 60 min and hold for about 51 hours with intermittent drying at −2° C. for 20 min ramp and 160 min hold,
vii) The vials are then stoppered in the presence of nitrogen gas, unloaded from Lyophilizer and sealed.

### DETAILED DESCRIPTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

In one aspect, the present invention provides a stable lyophilized composition of Cyclophosphamide prepared by a process comprising controlled lyophilization, wherein the annealing step is done at a temperature above 0° C.

In another aspect, the present invention provides a stable lyophilized composition of Cyclophosphamide that is easier to reconstitute and provides improved moisture content than the currently available lyophilized compositions of Cyclophosphamide.

In a preferred embodiment, the moisture content of the said lyophilized composition is not less than about 3.5% W/W, preferably from about 3.5% to 6% W/W.

In a preferred embodiment, the amount of Cyclophosphamide in the said stable lyophilized composition is in the range from 500 mg/vial to 2000 mg/vial.

The stable pharmaceutical composition of the present invention has sufficient stability to allow storage at a convenient temperature, preferably between −20° C. and 40° C., more preferably about 2° C. to about 25° C., for a reasonable period, e.g., the shelf-life of the product which can be as short as one month, but is typically three months, six months or longer, more preferably one year or two years. The composition of the present invention remains stable when stored at a temperature between 2° C. to 25° C., wherein the total impurities in the composition is not more than 5%. In particular, the lyophilized compositions remain stable at storage conditions of 25° C./60% RH and 2-8° C. for at least three months.

The nomenclature of known impurities is as mentioned below:
Cyclophosphamide related compound A: Bis(2-chloroethyl)amine hydrochloride,
Cyclophosphamide related compound B: 3-(2-Chloroethyl)-2-oxo-2-hydroxy-1,3,6,2-oxadiazaphosphonane,
Cyclophosphamide related compound C: 3-Aminopropyl dihydrogen phosphate,
Cyclophosphamide related compound D: 3-[2-(2-Chloroethylamino) ethylamino]propyl dihydrogen phosphate.

The lyophilization process, also known as freeze-drying technique is used to remove water from a solution to leave a dry ‘cake’ as an end product. In lyophilization, the water is removed from a product after it is frozen by placing it under vacuum, allowing the ice to change directly from solid to vapor, without passing through a liquid phase. The process consists of three separate and interdependent steps; namely freezing, primary drying and secondary drying. The term “controlled lyophilization” means controlling different parameters of lyophilization like cycle time/duration, temperature, vacuum, and other factors.

The inventors have successfully developed a process for preparation of stable lyophilized Cyclophosphamide composition having a moisture content not less than about 3.5% W/W.

Further the inventors of the present invention have surprisingly found that the annealing step during freezing phase of the lyophilization cycle results into uniform porous cake that is easier to reconstitute and having improved moisture content than the currently available lyophilized compositions of Cyclophosphamide.

The term “Annealing” is referred to as a process of transient increase in product temperature from initial set point to higher or lower set point, and then bringing the product temperature back to original set point. The Annealing step can be done on the product during different steps of freeze drying phase. For example, freezing the drug solution to −45° C. by ramping step and then holding at this temperature for specific time period, followed by raising temperature from about 2 to 15° C. and holding at this temperature for specific time period, and repeating such steps to get the desired product.

Further, the compositions of the present invention may comprise other pharmaceutically acceptable excipients selected from solvents, bulking agents, complexing agents, preservatives, anti-oxidants, stabilizers, tonicity modifiers or any other suitable excipients thereof.

The bulking agents for the purpose of the present invention include saccharides, preferably monosaccharides or oligosaccharides, sugar alcohols, and other suitable excipients thereof. The suitable bulking agents include the following, but are not limited to mannitol, sodium chloride, glucose, sucrose, lactose, trehalose, dextrose, maltose, sorbitol, dextran, raffinose, povidone, histidine and amino acids such as glycine, arginine, aspartic acid and mixtures thereof.

The lyophilized composition of the present invention may be reconstituted with sterile water for injection or other suitable solvents and further diluted with an intravenous admixture, such as normal saline. The pH of reconstituted solution is about 2.5 to 4. In a preferred embodiment, the reconstitution time of the composition is less than about 120 seconds, more preferably from about 60 to 80 seconds.

The stable cyclophosphamide composition of the present invention can be used in the treatment of diseases such as Hodgkin's disease, lymphocytic lymphoma, mixed-cell type lymphoma, histiocytic lymphoma, Burkitt's lymphoma, multiple myeloma, leukemias, mycosis fungoides, neuroblastoma, adenocarcinoma, retinoblastoma, Metastatic ovarian, breast carcinoma, Ewing's sarcoma, Small cell lung cancer and autoimmune diseases.

In another aspect, the present invention provides a process for the preparation of a stable lyophilized cyclophosphamide composition; wherein the process comprises the steps of:
i) Preparing a bulk solution comprising cyclophosphamide, mannitol, and water for injection,
ii) Filling the bulk solution into glass vials, which are then rubber stoppered,
iii) Loading the vials of step ii) into Lyophilizer at a temperature of about 5 to 25° C.,
iv) Performing the freezing step at a controlled temperature in the Lyophilizer, wherein annealing step is performed at a temperature from about 2° C. to 15° C., followed by freezing at about −45° C.
v) Setting the condenser temperature to −60° C. along with vacuum at 200 mtorr,
vi) Performing the primary drying at −18° C. with ramp for 60 min and hold for about 51 hours with intermittent drying at −2° C. for 20 min ramp and 160 min hold,
vii) The vials are stoppered in the presence of nitrogen gas, unloaded from Lyophilizer and sealed.

The conventional freeze-drying cycle is performed by the above mentioned process, excluding the annealing step of the step iv) in the manufacturing process.

In the following section, embodiments are described by a way of examples to illustrate the process of invention. However, these are not intended in any way to limit the scope of present invention.

### EXAMPLES

### Example 1: Stable Lyophilized Composition of Cyclophosphamide

| | | | Quantity | | |
|---|---|---|---|---|---|
| | | | (mg/ml) | | |
| Sr. | | | 500 | 1 | 2 |
| No. | Ingredients | Category | mg/vial | g/vial | g/vial |
| 1. | Cyclophos- | API | 534.5 mg | 1069 mg | 2138 mg |
| | phamide | | eq. to | eq. to | eq. to |
| | monohydrate | | 500 mg | 1000 mg | 2000 mg |
| | eq. to | | | | |
| | cyclophos- | | | | |
| | phamide | | | | |
| 2. | Mannitol | Bulking | 375 mg | 750 mg | 1500 mg |
| | | agent | | | |
| 3. | Nitrogen | Inert | q.s. to | q.s. to | q.s. to |
| | | gas | sparge | sparge | sparge |

### Manufacturing Process:

The pre-lyophilized solution for a stable cyclophosphamide composition can be prepared by following steps:
i) The water for injection is taken in manufacturing vessel; and nitrogen gas is sparged throughout the bulk formulation of cyclophosphamide.
ii) Cyclophosphamide is added to solution of step i) and stir to get a clear solution.
iii) Mannitol is added to the step ii) solution and stir to get a clear solution.
iv) The volume is adjusted with water for injection and stir to mix it properly.
v) The solution of step iv) is filtered through 0.2 micron sterilizing grade filter.
vi) The filtered bulk of step v) is filled into vials, and loaded in the Lyophilizer.

### Example 2: Annealing Step During Freezing Process in the Lyophilization Cycle

ANNEALING STEP DURING FREEZING PROCESS

| | Set Temp. (° C.) | | Estimated period | | Cycle Parameters |
|---|---|---|---|---|---|
| Loading step | 5° | C. | 30 | minutes | Hold |
| Freezing step | −10 to −45° | C. | 10-12 | hours | Ramp and Hold |
| Annealing step | 2 to 15° | C. | 5-6 | hours | Ramp and Hold |
| Freezing step | −45° | C. | 5-6 | hours | Ramp and Hold |

The annealing step during freezing process in the lyophilization cycle of cyclophosphamide can be performed as follows:
i) Loading the vials into Lyophilizer at a temperature of about 5 to 25° C.,
ii) Performing the freezing step at a controlled temperature in the Lyophilizer, wherein annealing step is performed at a temperature from about 2° C. to 15° C., followed by freezing at about −45° C.
iii) Setting the condenser temperature to −60° C. along with vacuum at 200 mtorr,
iv) Performing the primary drying at −18° C. with ramp for 60 min and hold for about 51 hours with intermittent drying at −2° C. for 20 min ramp and 160 min hold,
v) The vials are stoppered in the presence of nitrogen gas, unloaded from Lyophilizer and sealed.

### Example 3: Stability Data with Conventional Freeze-Drying Cycle for Lyophilized Cyclophosphamide as 500 mg/Vial

| | Rel. | Rel. | Rel. | Rel. | Single max | | | |
|---|---|---|---|---|---|---|---|---|
| | Com. | Com. | Com. | Com. | unknown | Total | % | Reconstitution |
| Condition | A | B | C | D | impurity | Impurity | Water | time |
| Initial | 0.024 | 0.268 | ND | 0.226 | 0.110% | 0.663% | 1.89% | 55 sec |
| 25° C., 1 M | 0.172 | 0.370 | ND | 0.931 | 2.215% | 4.704% | 1.43% | 35 sec |
| 2-8° C., 1 M | 0.028 | 0.253 | ND | 0.114 | 0.087% | 0.482% | 1.73% | 42 sec |
| 40° C., 1 M | 1.724 | 4.865 | 0.374 | 5.167 | 12.466% | 43.104% | 2.05% | 45 sec |
| 25° C., 2 M | 0.665 | 1.247 | 0.087 | 1.916 | 8.030% | 16.296% | 1.60% | 38 sec |
| 2-8° C., 2 M | 0.031 | 0.321 | ND | 0.344 | 0.325% | 1.146% | 2.44% | 39 sec |
| 25° C., 3 M | 0.319 | 0.187 | ND | 1.032 | 3.267% | 6.521% | 2.30% | 45 sec |
| 2-8° C., 3 M | 0.033 | 0.196 | ND | 0.236 | 0.136% | 0.655% | 3.24% | 48 sec |
| 25° C., 6 M | 0.692 | 0.435 | 0.186 | 1.999 | 7.292% | 15.906% | 4.06% | 48 sec |
| 2-8° C., 6 M | 0.049 | 0.165 | ND | 0.383 | 0.218% | 0.960% | 2.48% | 45 sec |

The conventional freeze-drying cycle is performed by the similar process as mentioned in the Example 2, excluding the annealing of the step ii) in the process.

The above results from a conventional freeze-drying cycle for Cyclophosphamide, that is freezing phase without annealing step, indicates poor stability in terms of higher impurities and lesser moisture content.

The nomenclature of known impurities is as mentioned below:
Cyclophosphamide related compound A: Bis(2-chloroethyl)amine hydrochloride,
Cyclophosphamide related compound B: 3-(2-Chloroethyl)-2-oxo-2-hydroxy-1,3,6,2-oxadiazaphosphonane,
Cyclophosphamide related compound C: 3-Aminopropyl dihydrogen phosphate,
Cyclophosphamide related compound D: 3-[2-(2-Chloroethylamino) ethylamino]propyl dihydrogen phosphate.

### Example 4: Stability Data for Lyophilized Cyclophosphamide as 500 mg/Vial

With Annealing at Temperature Above 0° C.

| | | | | | Single | | | |
|---|---|---|---|---|---|---|---|---|
| | Rel. | Rel. | Rel. | Rel. | max | | | |
| | Com. | Com. | Com. | Com. | unknown | Total | % | Reconstitution |
| Condition | A | B | C | D | impurity | impurity | Water | time |
| INITIAL | 0.026 | 0.088 | ND | 0.307 | ND | 0.421 | 4.22 | 60 sec |
| 25° C., 1 M | 0.039 | ND | 0.048 | 0.359 | 0.042 | 0.48 | 4.87 | 65 sec |
| 2-8° C., 1 M | 0.027 | 0.09 | ND | 0.248 | 0.059 | 0.424 | 5.94 | 65 sec |
| 40° C., 1 M | 0.089 | ND | 0.086 | 0.64 | 0.097 | 0.912 | 5.19 | 70 sec |
| 2-8° C., 2 M | 0.034 | 0.068 | 0.027 | 0.324 | ND | 0.453 | 4.57 | 75 sec |
| 25° C., 2 M | 0.05 | ND | 0.043 | 0.445 | 0.082 | 0.62 | 3.53 | 68 sec |
| 2-8° C., 3 M | 0.032 | 0.057 | ND | 0.375 | ND | 0.464 | 4.43 | 69 sec |
| 25° C., 3 M | 0.057 | ND | 0.033 | 0.429 | 0.088 | 0.607 | 4.11 | 65 sec |

The above results from a controlled lyophilization cycle with annealing step, indicates enhanced stability in terms of lesser impurities and improved moisture content than the conventional lyophilized compositions of Cyclophosphamide. The product obtained from lyophilization cycle is a white cake in a clear glass vial.

### Example 5: Composition of Cyclophosphamide Injection; 500 mg/Vial and 1000 Mg/Vial

| Sr. No. | Ingredients | Quantity (mg/ml) |
|---|---|---|
| 1 | Cyclophosphamide | 35.00 |
| 2 | Mannitol (Pearlitol PF) | 26.25 |
| 3 | Water for Injection | q.s. to 1 ml |
| 4 | Nitrogen | q.s. to sparge |

### Manufacturing Process:

i) Sparging the nitrogen gas throughout the bulk solution of Cyclophosphamide for injection,
ii) Adding mannitol into the solution, and stirring for about 10 minutes,
iii) Adjusting the volume with water for injection, and stirring for about 10 minutes,
iv) Filtering the bulk solution through the sterilizing grade filter,
v) Filling the volume of 14.57 ml of filtered bulk solution into 30 ml glass vials to make up cyclophosphamide 500 mg/vial.
vi) Filling the volume of 29.14 ml of filtered bulk solution into 30 ml glass vials to make up cyclophosphamide 1000 mg/vial.
vii) The filled vials are stoppered, and loaded into the Lyophilizer.

### Example 6: Annealing Step During Freezing Process in Lyophilization Cycle for Cyclophosphamide Injection 500 mg/Vial

ANNEALING STEP DURING FREEZING PROCESS

| | Set Temp. (° C.) | | Estimated period | | Cycle Parameters |
|---|---|---|---|---|---|
| Loading step | 5° | C. | 35 | minutes | Ramp and Hold |
| Freezing step | −10 to −40° | C. | 10-12 | hours | Ramp and Hold |
| Annealing step | 2 to 15° | C. | 4-5 | hours | Ramp and Hold |
| Freezing step | −45° | C. | 5-6 | hours | Ramp and Hold |

### Example 7: Annealing Step During Freezing Process in Lyophilization Cycle for Cyclophosphamide Injection 1000 mg/Vial

ANNEALING STEP DURING FREEZING PROCESS

| | Set Temp. (° C.) | | Estimated period | | Cycle Parameters |
|---|---|---|---|---|---|
| Loading step | 5° | C. | 31 | minutes | Ramp and Hold |
| Freezing step | −10 to −40° | C. | 11-13 | hours | Ramp and Hold |
| Annealing step | 2 to 15° | C. | 5-7 | hours | Ramp and Hold |
| Freezing step | −45° | C. | 5-6 | hours | Ramp and Hold |

In the above Examples 6 and 7, the annealing step in lyophilization cycle of cyclophosphamide can be performed as follows:
i) Loading the vials into Lyophilizer at a temperature of about 5 to 25° C.,
ii) Performing the freezing step at a controlled temperature in the Lyophilizer, wherein annealing step is performed at a temperature from about 2° C. to 15° C., followed by freezing at about −45° C.
iii) Setting the condenser temperature at −60° C. along with vacuum at 200 mtorr,
iv) Performing the primary drying at −18° C. with ramp for 60 min and hold for about 51 hours with intermittent drying at −2° C. for 20 min ramp and 160 min hold,
v) The vials are stoppered in the presence of nitrogen gas, unloaded from Lyophilizer and sealed.

### Example 8: Stability Studies of Cyclophosphamide for Injection 500 mg/Vial & 1000 mg/Vial at 25° C./60% RH and 2-8° C. Storage Conditions

| | | | | | | | | |
|---|---|---|---|---|---|---|---|---|
| | Rel. | Rel. | Rel. | Rel. | | | | |
| | Com. | Com. | Com. | Com. | Total | % | | Reconstitution |
| Condition | A | B | C | D | Impurity | Water | Assay | time |

500 mg/vial

| | | | | | | | | |
|---|---|---|---|---|---|---|---|---|
| Initial | 0.010 | 0.066 | ND | 0.211 | 0.287% | 4.81% | 100.6 | 62 sec |
| 25° C., 3 M | 0.044 | ND | 0.052 | 0.355 | 0.474% | 3.75% | 100.2 | 57 sec |
| 25° C., 6 M | 0.070 | ND | 0.059 | 0.409 | 0.558% | 3.90% | 99.1 | 55 sec |
| 2-8° C., 3 M | 0.011 | 0.022 | ND | 0.165 | 0.198% | 3.85% | 100.8 | 49 sec |
| 2-8° C., 6 M | 0.011 | 0.035 | ND | 0.125 | 0.171% | 5.23% | 100.0 | 60 sec |

1000 mg/vial

| | | | | | | | | |
|---|---|---|---|---|---|---|---|---|
| Initial | 0.011 | 0.126 | ND | 0.060 | 0.197% | 5.61% | 100.2 | 51 sec |
| 25° C., 3 M | 0.047 | 0.037 | 0.035 | 0.204 | 0.396% | 4.63% | 100.9 | 60 sec |
| 2-8° C., 3 M | 0.012 | 0.106 | ND | 0.091 | 0.275% | 5.59% | 99.7 | 55 sec |
| 2-8° C., 6 M | 0.018 | 0.062 | ND | 0.129 | 0.209% | NA | 101.4 | 57 sec |

Based on the review of stability data of Cyclophosphamide for Injection (500 mg/vial and 1000 mg/vial), it can be concluded that product is stable at 2-8° C. and 25° C./60% RH storage conditions. Therefore, the developed formulations of Cyclophosphamide for Injection 500 mg/vial and 1000 mg/vial are considered to be stable at the proposed storage conditions for at least three months.

The detailed description and the example provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

We claim:

1. A stable lyophilized composition of Cyclophosphamide prepared by a process comprising controlled lyophilization, wherein the annealing step is done at a temperature above 0° C.

2. The stable lyophilized composition of Cyclophosphamide according to claim 1, wherein the process comprises controlled lyophilization with the annealing step at a temperature of about 2° C. to 15° C.

3. The stable lyophilized composition of Cyclophosphamide according to claim 1, wherein the amount of Cyclophosphamide is from 500 mg/vial to 2000 mg/vial.

4. The stable lyophilized composition of Cyclophosphamide according to claim 1, wherein the composition comprises mannitol as a bulking agent.

5. The stable lyophilized composition of Cyclophosphamide according to claim 1, wherein moisture content of the composition is not less than about 3.5% W/W.

6. The stable lyophilized composition of Cyclophosphamide according to claim 1, wherein the composition can be reconstituted in less than about 120 seconds.

7. The stable lyophilized composition of Cyclophosphamide according to claim 1, wherein the total impurity in the composition is not more than 5%, when stored at a temperature between 2-8° C. and 25° C./60% RH for at least three months.

8. A process for preparation of a stable lyophilized composition comprising the steps of:
i) Preparing a bulk solution comprising cyclophosphamide, mannitol, and water for injection,
ii) Filling the bulk solution into vials, which are then rubber stoppered,
iii) Loading the vials of step ii) into Lyophilizer at a temperature of about 5 to 25° C.,
iv) Performing the freezing step at a controlled temperature in the Lyophilizer, wherein annealing step is performed at a temperature from about 2° C. to 15° C., followed by freezing at about −45° C.,
v) Performing the drying step to produce the stable lyophilized composition.

9. A stable lyophilized composition of Cyclophosphamide, wherein the moisture content of the composition is not less than about 3.5% W/W, and wherein the composition remains stable for at least three months upon storage at a temperature between 2° C. to 25° C.

10. A stable lyophilized composition of Cyclophosphamide, wherein the moisture content of the composition is not less than about 3.5% W/W, and the composition can be reconstituted in less than about 120 seconds.
